(19) United States
(12) Patent Application Publication
(10) Pub. No.: US 2007/0219619 A1
(43) Pub. Date: Sep. 20, 2007

(54) PARTIALLY COVERED STENT DEVICES AND METHODS OF USE

(75) Inventors: Martin S. DIECK, Cupertino, CA (US); Nicholas C. DEBEER, Montara, CA (US)

Correspondence Address: LEVINE BAGADE HAN LLP, 2483 EAST BAYSHORE ROAD, SUITE 100, PALO ALTO, CA, 94303, US

(73) Assignee: Cardiovasc, Inc., Menlo Park, CA (US)

(21) Appl. No.: 11/553,900

(22) Filed: Oct. 27, 2006

Related U.S. Application Data
(60) Provisional application No. 60/730,979, filed on Oct. 27, 2005.

(51) Int. Cl. A61F 2/06

(52) U.S. Cl. 623/1.13; 623/1.16

(57) ABSTRACT

Devices, systems and methods are provided for treating aneurysms, particularly cerebral aneurysms. Such treatment is achieved minimally invasively without the need for conventional filling materials and methods. Such treatments may be used for aneurysms located near blood vessel side-branches and bifurcations.

### CROSS-REFERENCES TO RELATED APPLICATIONS

This application claims priority to U.S. Provisional Patent Application No. 60/730,979 filed Oct. 27, 2005, incorporated herein by reference for all purposes.

### BACKGROUND OF THE INVENTION

A cerebral aneurysm is an area where a blood vessel in the brain weakens, resulting in a bulging or ballooning out of part of the vessel wall. The disorder may result from congenital defects or from other conditions such as high blood pressure, atherosclerosis or head trauma. Every year, an estimated 30,000 people in the United States experience a ruptured cerebral aneurysm, and up to 6 percent of the population may be living with an unruptured aneurysm. Aneurysms occur in all age groups, but the incidence increases steadily for individuals age 25 and older, is most prevalent in people ages 50 to 60, and about three times more prevalent in women. The outcome for patients treated before a ruptured aneurysm is much better than for those treated after, so the need for adequate treatment of a cerebral aneurysm is very important.

Current treatment options include a surgical operation to “clip” the aneurysm which is performed by doing a craniotomy, and isolating the aneurysm from the bloodstream using one or more clips, which allows it to deflate. Surgical repair of cerebral aneurysms is not possible if they are located in unreachable parts of the brain. Angiography is used to visualize closure of the aneurysm and preserve normal flow of blood in the brain.

A less invasive technique which does not require surgery, called endovascular therapy, uses micro catheters to deliver coils to the site of the enlarged blood vessel that occludes the aneurysm from inside the blood vessel. In some cases, the aneurismal opening or neck is too large to retain these coils. In such cases, a stent may be used to create a bridge across the neck and prevent the coils from encroaching into the vessel lumen. Typically, such a stent comprises a small flexible cylindrical mesh tube that provides a scaffolding to assist in holding the coils in place. An example of such a stent is provided by Neuroform3™ Microdelivery Stent System (Boston Scientific, Inc.). Neuroform3 Stents employ a highly flexible, hybrid cell design for better tracking during access and greater conformability within a variety of vessel morphologies. The Neuroform3 hybrid cell design is also engineered to provide greater scaffolding for coil mass support and sufficient radial force to generate stability within the vessel. However, the Neuroform3 Stents are only used to hold the coils in place and cannot be used independently to treat aneurysms.

Therefore, a stent design is desired that is useable itself for treatment of an aneurysm without the need for filling material, such as coils. Therefore, such a stent may be used to treat aneurysms which are typically unsuitable for filling with material. Such a stent design should provide high flexibility for deliverability through tortuous cerebral anatomy and for conformability within a variety of vessel morphologies while providing sufficient radial strength to hold the stent firmly in place. Such a stent design should also be useable to treat aneurysms located near blood vessel side-branches and bifurcations. At least some of these objectives will be fulfilled by the present invention.

### BRIEF SUMMARY OF THE INVENTION

Devices, systems and methods are provided for treating aneurysms, particularly cerebral aneurysms. Such treatment is achieved minimally invasively without the need for conventional filling materials and methods. Such treatments may be used for aneurysms located near blood vessel side-branches and bifurcations.

In a first aspect of the present invention, a stent device is provided for covering an aneurysm in a blood vessel, particularly wherein the blood vessel includes at least one side-branch near the aneurysm. In some embodiments, the stent device comprises a tubular frame having a first end and a second end, and a covering between the first and second ends of the frame. The covering substantially restricts flow of blood through the frame to the aneurysm while the stent device is positioned within the blood vessel so that the covering substantially covers the aneurysm. Also, the tubular frame has a cell geometry which allows sufficient flow of blood through the cell geometry at least between the ends and the covering so as to maintain blood flow through to the at least one side-branch. The frame typically also includes at least one anchoring portion which provides radial anchoring force.

The covering partially occludes, blocks or covers the frame so as to restrict the flow therethrough, i.e. in a lateral direction through the wall of the stent. The covering may cover any suitable portion of the frame, such as approximately 10-90 percent of the frame or more particularly approximately 30-40 percent of the frame. Such percentages may be in length of the frame covered or in area of the frame covered. In some embodiments, the covering is positioned approximately equidistant from the ends. In other embodiments, the covering is positioned at one of the first end or the second end.

The covering may have a variety of shapes, sizes, materials and configurations as will be described in more detail herein. For example, the covering may comprise an expandable polymer material. Or the covering may be woven through the cell geometry of the frame, such as in a spiral configuration. In some embodiments, the covering comprises a tubular sleeve positionable around the frame. In these embodiments in particular, the device may also include at least one security ring configured to assist in holding the covering on the frame.

In another embodiment, the stent device comprises a tubular frame having a first end, a second end, an occlusional cell geometry and an open cell geometry. The occlusional cell geometry is configured to be positioned so as to cover the aneurysm and substantially prevent flow of blood therethrough to the aneurysm while the stent device is positioned within the blood vessel. The open cell geometry is configured to be positioned so as to maintain blood flow through to the at least one side-branch while the stent device is positioned within the blood vessel. Typically, the occlusional cell geometry has smaller cells than the open cell geometry. In some embodiments, the occlusional cell geometry comprises approximately 10-90 percent of the frame, particularly approximately 30-40 percent of the frame.

In some instances, the occlusional cell geometry is disposed approximately equidistant from the ends. The frame may also include at least one anchoring portion which provide radial anchoring force.

In another aspect of the present invention, a method is provided for covering an aneurysm in a blood vessel, particularly wherein the blood vessel includes at least one side-branch near the aneurysm. In one embodiment, the method includes advancing a stent through the blood vessel, wherein the stent comprises a tubular frame having a first end, a second end, an open cell geometry and a covering. The method also includes positioning the stent within the blood vessel so that the covering substantially covers the aneurysm restricting blood flow to the aneurysm and the open cell geometry substantially covers the at least one side-branch allowing blood flow to the at least one side-branch.

When the covering is disposed approximately equidistant from the ends, positioning may comprise positioning the ends on opposite sides of the aneurysm. When the stent includes an anchoring portion near the first end and the covering near the second end and when the blood vessel includes a bifurcation near the aneurysm, positioning may comprise positioning the anchoring portion within the blood vessel so as to anchor the stent while the second end is disposed near the bifurcation.

In addition, positioning the stent typically comprises expanding the stent within the blood vessel. Such methods are often performed when the blood vessel comprises a cerebral blood vessel but are not so limited. Other objects and advantages of the present invention will become apparent from the detailed description to follow, together with the accompanying drawings.

### BRIEF DESCRIPTION OF THE DRAWINGS

FIG. 1 illustrates a perspective view of an embodiment of a stent of the present invention.

FIG. 2 illustrates the stent of FIG. 1 positioned within a blood vessel having an aneurysm.

FIG. 3 illustrates an embodiment of a frame.

FIG. 4 illustrates a covering asymmetrically positioned over a frame.

FIG. 5 illustrates the stent of FIG. 4 positioned within blood vessel having an aneurysm near a bifurcation.

FIGS. 6A-6D illustrate an embodiment of the stent of the present invention.

FIGS. 7A-7C illustrate embodiment of the stent of the present invention wherein the frame has a variable density.

FIG. 8 illustrates another embodiment of a stent wherein the frame has a higher density toward the ends and a lower density therebetween.

FIGS. 9A-9B illustrate an embodiment of a stent wherein the frame has circular belts near the ends comprised of a plurality of elongate struts in a zig-zag arrangement.

FIG. 10A-10C illustrate an embodiment of a stent having a covering that is woven through the frame so that the covering wrapped on itself.

FIG. 11 illustrates an embodiment of a stent having a covering that is woven through the frame so that the covering is wrapped in a spiral configuration.

FIG. 12 illustrates an embodiment of stent for use without a separate covering.

### DETAILED DESCRIPTION OF THE INVENTION

FIG. 1 illustrates an embodiment of a stent 10 of the present invention. In this embodiment, the stent 10 comprises a frame 12, a graft or covering 14, and a pair of security rings 16. The frame 12 has a tubular shape and extends from a first end 18 to a second end 20. The covering 14 is sized to cover a portion of the frame 12, typically approximately ⅓ of the length of the frame 12. In this embodiment, the covering 14 is positioned over the exterior of the frame 12 and secured in place by the security rings 16 which are positioned thereon. Additional description and embodiments are provided in later sections.

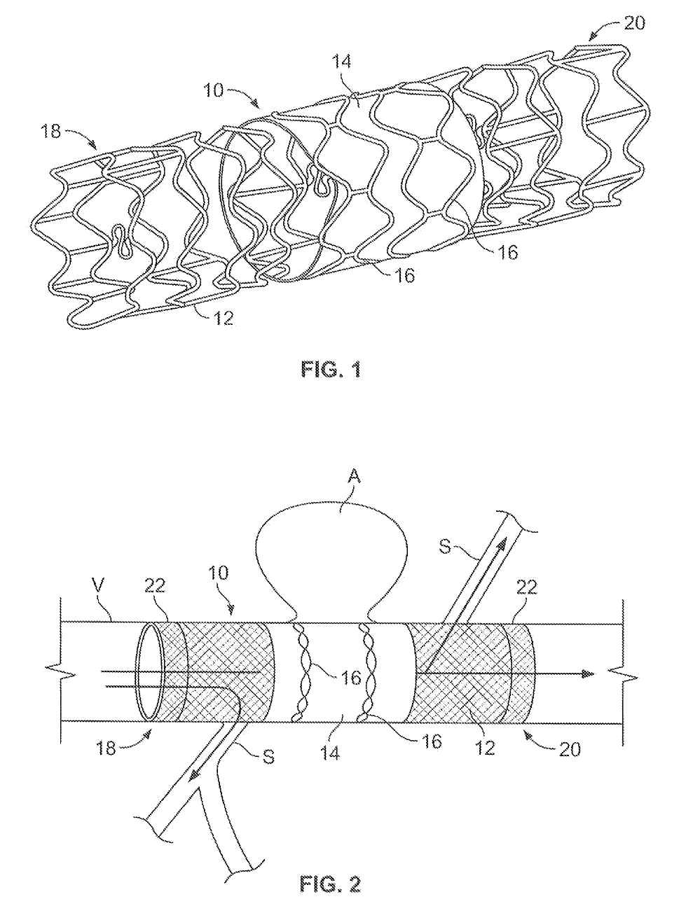

FIG. 2 illustrates the stent 10 of FIG. 1 positioned within a blood vessel V having an aneurysm A. As shown, the stent 10 is positioned so that the covering 14 covers the opening of neck of the aneurysm A, restricting blood flow into the aneurysm A. Thus, the aneurysm A is excluded from the circulation without the need for filing the aneurysm A, such as with coils. In this example, the blood vessel V also has side-branches S which are located relatively close to the aneurysm A. The covering 14 is positioned so as to substantially avoid covering the side-branches S and allow continued blood flow into the side-branches S, as illustrated by arrows. In this embodiment, the ends 18, 20 of the frame 12 are positioned on opposite sides of the aneurysm A and side-branches S. Thus, the frame 12 extends over the side-branches, however, the frame 12 has an open cell geometry which allows adequate flow into the side-branches through the frame 12. In this embodiment, the frame 12 also includes anchoring portions 22 near each end 18, 20 wherein the cell geometry is provides a higher radial strength. This assists in anchoring the stent 10 within the blood vessel V.

The frame 12 may have a variety of configurations. Example embodiments of frames 12 are provided in U.S. Pat. Nos. 6,371,980; 6,451,050; 6,520,984 and PCT/US2006/031059, each of which are incorporated herein by reference for all purposes. The frame 12 is expandable from a contracted, small-diameter condition to a radially expanded condition under the influence of an expanding force, typically an expandable balloon catheter used in delivering and placing the device in a blood vessel, according to conventional stent placement methods. Alternatively, the stent may be self-expanding. In some embodiments, the frame 12 has a length in the range of approximately 5-30 mm, particularly in the range of approximately 8-20 mm. Likewise, in some embodiments, the frame 12 has an outer diameter in the range of approximately 1-10 mm, particularly in the range of approximately 2.5-6 mm.

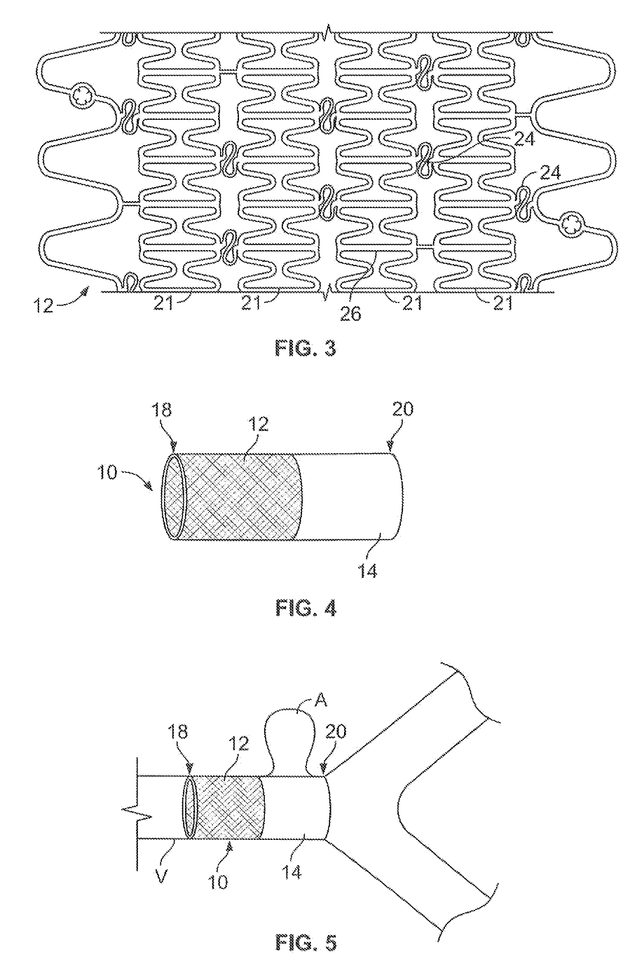

An example of such a frame 12 is illustrated in FIG. 3. As shown, the frame 12 has a plurality of axially spaced-apart circular belts 21 which are interconnected by interconnectors 24. Each belt 21 is comprised of a plurality of circumferentially spaced-apart elongate struts 26. The interconnectors 24 adjoin the ends of the struts 26 and form in conjunction therewith the circular belts 21. The interconnectors 24 are disposed at circumferentially spaced-apart positions to provide circumferential support when the stent is expanded while at the same time being axially flexible. In preferred embodiments, the interconnectors 42 are sinusoidal or serpentined shaped which assist in allowing expansion.

The frame 12 may be formed from any suitable method. For example, the frame 12 may be comprised of a tube having a desired pattern formed or cut therefrom, such as by laser cutting or chemical etching. Alternatively, the desired pattern may be formed out of a flat sheet, e.g. by laser cutting or chemical etching, and then rolling that flat sheet into a tube and joining the edges, e.g. by welding. Further, the frame 12 may be formed by etching a pattern into a material or mold and depositing stent material in the pattern, such as by chemical vapor deposition or the like. Or the frame 12 may be formed from a weave or braid. Any other suitable manufacturing method known in the art may be employed for manufacturing a frame in accordance with the invention.

The frame 12 may be comprised of plastic, metal or other materials and may exhibit a multitude of configurations. Example plastics include polyurethanes and polycarbonates. Example metals include 316LVM, L605 Cobalt Chromium, stainless steel, titanium, Nitinol, and tantalum among others. The frame 12 may also be treated to improve biocompatibility, such as by electropolishing or polymer coating.

It may be appreciated that the frame 12 may have a variety of other forms, including conventional stents, coils, wireframes, etc.

Typically, the covering 14 has a tubular shape configured to fit over the frame 12. However, the covering 14 may alternatively be disposed under the frame 12 and attached thereto. Thus, the covering 14 is also expandable from a contracted, small-diameter condition to a radially expanded condition. This may be achieved by constructing the covering 14 from a flexible material, such as a polymer. Example materials include expandable polymer material, e.g., a porous or non-porous polytetrafluoroethylene (PTFE) material. Alternatively, this may be achieved by movement of the covering 14 as the frame 12 expands, such as by reducing overlap of the covering 14.

An exemplary covering 14 is described in U.S. Pat. No. 6,371,980, issued Apr. 16, 2002, and in PCT/US2006/031059, each of which are incorporated by reference herein in their entirety. It may be appreciated that the covering 14 may have a variety of other forms, including conventional sleeves, spirals or helixes. Also, the covering 14 may cover a side of the frame 12, rather than extending around the frame 12.

In the present invention, the covering 14 covers only a portion of the frame 12, preferably approximately 10-90% of the frame 12, more preferably approximately 30-40% of the frame 12. The covering 14 may be symmetrically positioned over the frame 12, such as equidistant from the ends 18, 20, as illustrated in FIG. 2. Or, the covering 14 may be asymmetrically positioned, such as covering at least part of end 18 or end 20 but not both, as illustrated in FIG. 4. Such asymmetrical positioning may be useful when treating aneurysms located near a bifurcation in a blood vessel V, such as illustrated in FIG. 5. Here, the covering 14 covers the aneurysm A and end 18 of the frame 12 is secured within the blood vessel. However, the bifurcation on the opposite side of the aneurysm A is not conducive to anchoring therein so the stent 10 is primarily secured in place by end 18.

In some embodiments, the stent 10 includes one or more clips or security rings 16 which are used to secure the covering 14 to the frame 12, such as illustrated in FIGS. 1-2. Exemplary security rings 16 are described in U.S. patent application Ser. No. 10/255,199, filed Sep. 26, 2002, and PCT/US2006/031059, each of which are incorporated by reference herein in their entirety. In order to ensure that the covering 14 remains in the desired position on the frame 12, security rings 16 are positioned over the covering 14, such as over the outer ends of the covering 14. Alternatively, the rings 16 may be positioned inside or within the frame 12, such as when the covering is within the frame 12. The security rings 16 may be formed of a metal and preferably the same metal which is used for the frame 12, or the rings 16 may be comprised of other suitable material, such as a polymer. By way of example, the security rings 16 can be formed from laser cut tubing in the same manner as some embodiments of the frame 12 having a suitable wall thickness of 0.003″ to 0.006″. The inner surfaces of the security rings 16 can be left unpolished so that they have a rougher inner surface finish to enhance gripping to the outer surface of the covering 14. Alternatively, a texture can be applied to the inner surface to enhance the gripping capabilities of the security ring 16.

The rings 16 may have a variety of shapes, including sinusoidal-shaped convolutions so that they can be expanded with the frame 12 and covering 14. The security rings 16 can be placed at any location along the covering 14 including partially over the covering 14 and partially over the frame 12 itself. Optionally, the rings 16 may also include at least one radiopaque marker. In addition, spun FEP or polymer may be used to hold the rings 16 in place or create a smooth transition between the rings and the frame or covering.

It may be appreciated that other structures may be employed in the stent 10 for anchoring the covering 14 on the frame 12. For example, the covering 14 could be sewn on the frame 12 or bonded to the frame 12 by polymer welds, urethane, spun fiber or the like.

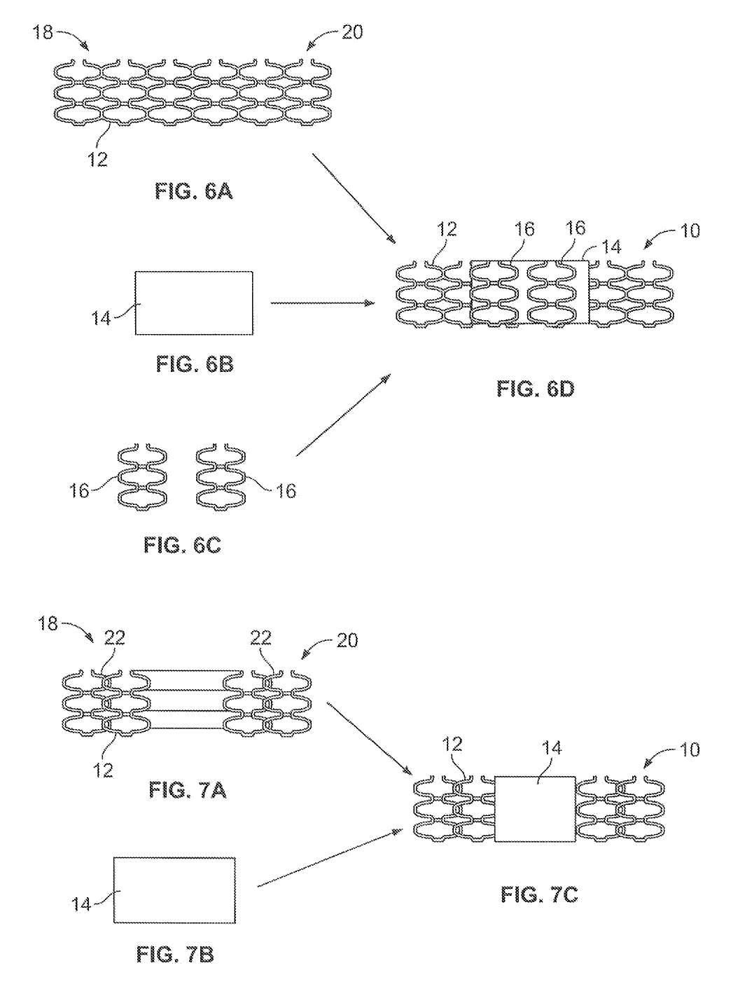

FIGS. 6A-6D illustrate an embodiment of the stent 10 of the present invention. As shown, the stent 10 comprises a frame 12 (FIG. 6A), a covering 14 (FIG. 6B), and at least one security ring 16 (FIG. 6C). FIG. 6D illustrates the assembled stent 10 wherein the covering 14 is positioned over the frame 12 symmetrically between the ends 18, 20. Also, the security rings 16 are placed over the covering 14 to hold the covering in place.

FIGS. 7A-7C illustrate another embodiment of the stent 10 of the present invention. Here the stent 10 comprises a frame 12 (FIG. 7A) that has a variable density. The density of the frame 12 toward the ends 18, 20 increases so as to provide higher radial strength. Thus, these areas may be considered anchoring portions 22. The density of the frame 12 decreases toward the center so as to provide sufficient support the covering 14 yet allow adequate flexibility and flow therethrough so as to avoid occluding side-branches of a blood vessel. A lower density portion of the frame 12 may have a more open cell geometry wherein the cells are larger. Or, the percentage of open space may be larger. FIG. 7A shows the lower density portion of the frame 12 to have longitudinal struts extending between the ends 18, 20. FIG. 7C 6D illustrates the assembled stent 10 wherein the covering 14 is positioned over the frame 12 symmetrically between the ends 18, 20.

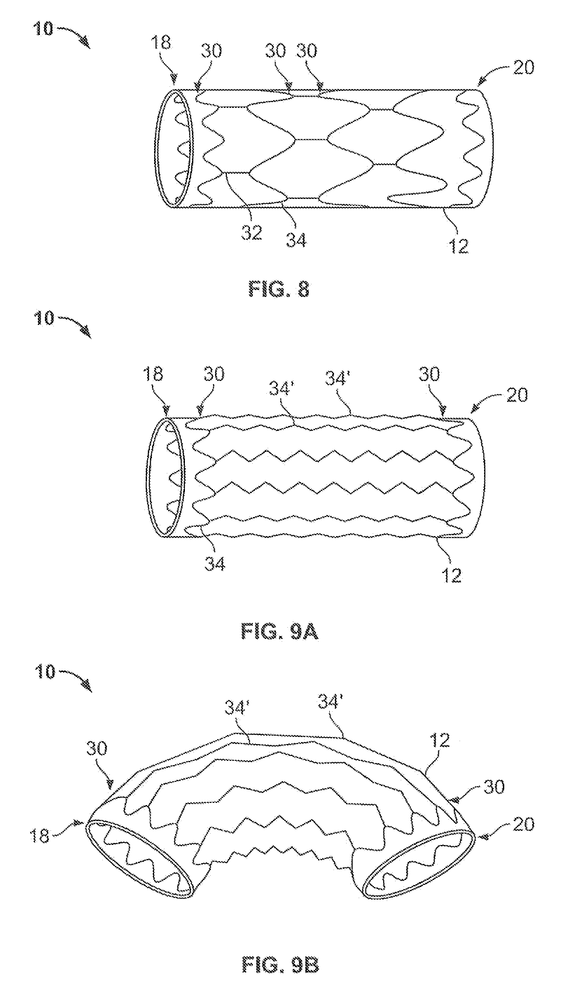

FIG. 8 illustrates another embodiment of a stent 10 wherein the frame 12 has a higher density toward the ends 18, 20 and a lower density therebetween. In this embodiment, the frame 12 has a plurality of axially spaced-apart circular belts 30 which are interconnected by interconnectors 32. Each belt 30 is comprised of a plurality of elongate struts 34 in a “zig-zag” arrangement. The interconnectors 32 adjoin the ends of the struts 34. In this embodiment, belts 30 near the ends 18, 20 have a shorter strut length (e.g. 0.020-0.100 inches, preferably 0.070-0.090 inches) than belts 30 therebetween having a longer strut length (e.g. 0.070-0.200 inches, preferably 0.100-0.120 inches). Thus, the belts 30 near the ends 18, 20 have a higher density and therefore higher radial strength while the belts 30 therebetween have a lower density and therefore lower stiffness (higher flexibility).

FIGS. 9A-9B illustrate another embodiment of a stent 10 wherein the frame 12 has a higher density toward the ends 18, 20 and a lower density therebetween. In this embodiment, the frame 12 has circular belts 30 near the ends 18, 20 comprised of a plurality of elongate struts 34 in a zig-zag arrangement. The belts 30 are joined by longitudinal struts 34′ that extend between the ends 18, 20. The longitudinal struts 34′ have a zig-zag or accordion shape. The circular belts 30 near the ends 18, 20 provide sufficient radial strength for anchoring of the stent 10 within a blood vessel. And, the longitudinal struts 34′ provide sufficient support for a covering yet a low enough density to allow passage therethrough of blood flow into side-branches of the blood vessel. In addition, the accordion shape of the longitudinal struts 34′ allows for higher bending and flexibility through tortuous anatomy. For example, FIG. 9B shows the stent 10 of FIG. 9A positioned in a curved or bent configuration as may occur when passing through the vasculature, particularly the cerebral vasculature. The accordion shape of the longitudinal struts 34′ allows for some struts 34′ to extend while other struts 34′ contract. Thus, the struts 34′ resist fatigue and allow higher flexibility of the stent 10.

It may be appreciated that the embodiments of stents 10 illustrated in FIG. 8 and FIGS. 9A-9B typically include a covering positioned over a portion of the stent 10. In preferred embodiments, the covering is positioned between the ends 18, 20 and is supported by the lower density portion of the frame 12. Optionally, the covering is held in place by security rings.

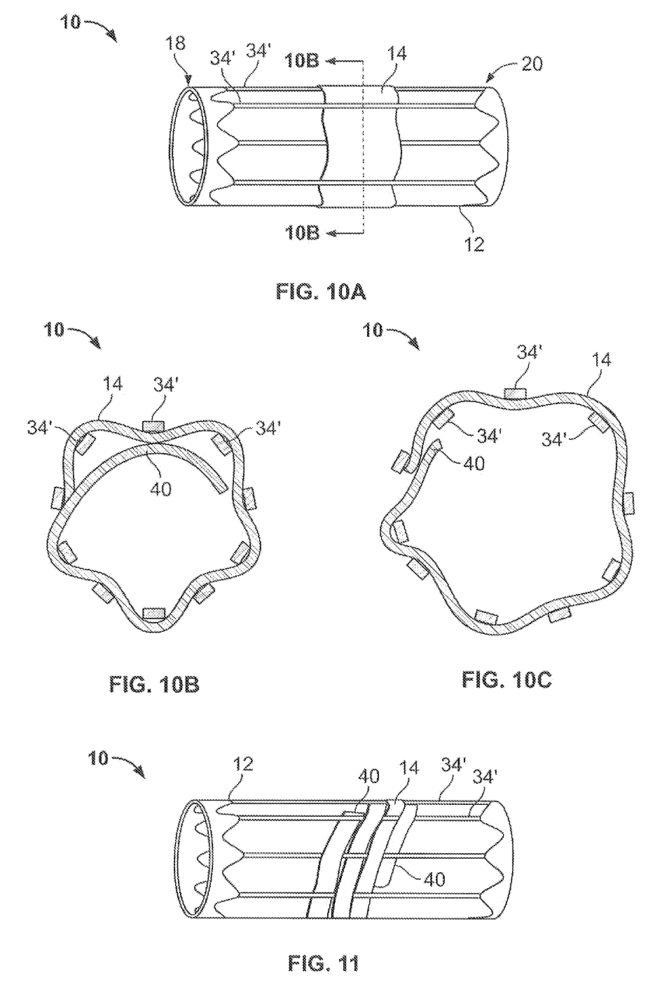

In some embodiments, the covering 14 is woven through the frame 12 so that the covering 14 is substantially held in place by such weaving. An example of such an embodiment is illustrated in FIG. 10A. In this embodiment, the frame 12 comprises longitudinal struts 34′ through which the covering 14 is woven circumferentially around the frame 12. As shown, the covering 14 has a ribbon shape and alternates passing over and under the individual struts 34′. The covering 14 can also be placed in any position between the ends 18, 20. FIGS. 10B-10C illustrate a cross-sectional view of the woven covering 14 of FIG. 10A. FIG. 10B shows the stent 10 in an unexpanded position having a smaller diameter. In this position, the covering 14 is wrapped on itself, as illustrated by a free end 40 of the covering 14 extending circumferentially within the frame 12. FIG. 10C shows the stent 10 in an expanded position having a larger diameter. As the stent 10 expands, the covering 14 is pulled outwardly with the expanding frame 12 and the covering at least partially unwraps, as illustrated by the free end 40 of the covering 14 extending less within the frame 12. In some embodiments, as illustrated in FIG. 11, the covering 14 is woven circumferentially around the frame 12 in a spiral fashion. Thus, rather than the covering 14 wrapping on itself, the free ends 14 are pulled around the frame 12 as the frame expands.

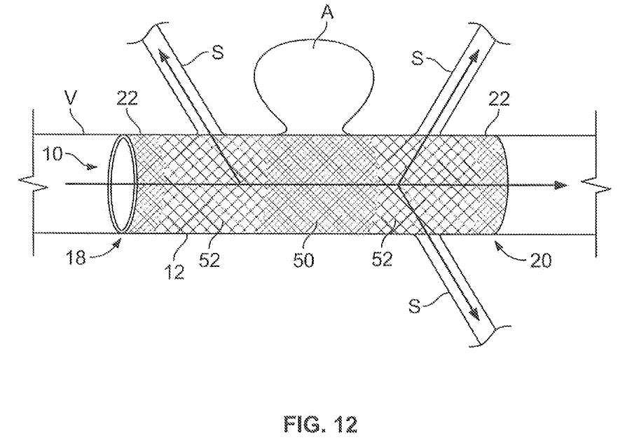

It may be appreciated that in some embodiments, a separate covering is not used; rather, portions of the frame 12 itself act as the “covering” so as to block or restrict flow into the aneurysm. Such portions may be considered to have an occlusional cell geometry. An example of such a stent 10 is illustrated in FIG. 12. As shown, the stent 10 is comprised of a frame 12 having a variety of densities for various purposes. For example, the frame 12 includes higher density areas near the ends 18, 20 for anchoring (anchoring portions 22), a higher density area positionable over the aneurysm A to block flow therethrough (occlusional cell geometry 50), and lower density areas therebetween (open cell geometry 52) for flexibility and passage of flow therethrough into side-branches S of the blood vessel V. Densities may be controlled by strut length, interconnector length, etc. In addition, radial strength and flexibility may be controlled by strut thickness or cross-sectional dimensions. In some embodiments, typical strut cross-sectional dimension is approximately 0.006 in. by 0.006 in. square. Some portions of the frame 12 may have thinner cross-sections, such as approximately 0.004 in. by 0.004 in. square, to provide higher flexibility. Other portions of the frame 12 may have thicker cross-sections, such as approximately 0.010 in. by 0.010 in. square, to provide higher radial force. Thus, strut cross-sectional dimension may be varied to provide differing stent characteristics.

In any of the above embodiments, the cell geometry of the frame 12 may be altered in desired areas to accommodate particular anatomies. For example, to ensure adequate flow to a side-branch, the frame 12 may be altered in the area of the side-branch to increase flow therethrough. This may be achieved by widening the cell geometry in this area, typically the lower density area or open cell geometry area. To widen a desired cell, an inflatable member or balloon may be passed through the desired cell and expanded to change its dimensions (e.g. cause widening). When the frame 12 is comprised of a weave or braid, the struts move apart according to the weave. When the frame 12 is comprised of a cut tube or sheet, the struts may be deformed upon widening of the cells. Any number of cells may be widened in any location to achieve the desired result. It may be appreciated that widening in some areas may cause constriction or narrowing in other areas which may be utilized for various purposes. For example, widening in an open cell geometry area may cause narrowing in the occlusional cell geometry area which may benefit the overall stent design.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A stent device for covering an aneurysm in a blood vessel, wherein the blood vessel includes at least one side-branch near the aneurysm, the device comprising:
a tubular frame having a first end and a second end; and
a covering between the first and second ends of the frame which substantially restricts flow of blood through the frame to the aneurysm while the stent device is positioned within the blood vessel so that the covering substantially covers the aneurysm, and
wherein the tubular frame has a cell geometry which allows sufficient flow of blood through the cell geometry at least between the ends and the covering so as to maintain blood flow through to the at least one side-branch.

2. A device as in claim 1, wherein the covering covers approximately 10-90 percent of the frame.

3. A device as in claim 1, wherein the covering covers approximately 30-40 percent of the frame.

4. A device as in claim 1, wherein the covering is positioned approximately equidistant from the ends.

5. A device as in claim 1, wherein the covering is positioned at one of the first end or the second end.

6. A device as in claim 1, wherein the covering comprises a tubular sleeve positionable around the frame.

7. A device as in claim 6, further comprising at least one security ring configured to assist in holding the covering on the frame.

8. A device as in claim 1, wherein the covering comprises an expandable polymer material.

9. A device as in claim 1, wherein the covering is woven through the cell geometry of the frame.

10. A device as in claim 9, wherein the covering is woven in a spiral configuration.

11. A device as in claim 1, wherein the frame includes at least one anchoring portion which provides radial anchoring force.

12. A stent device for covering an aneurysm in a blood vessel, wherein the blood vessel includes at least one side-branch near the aneurysm, the device comprising:
a tubular frame having a first end, a second end, an occlusional cell geometry and an open cell geometry,
wherein the occlusional cell geometry is configured to be positioned so as to cover the aneurysm and substantially restrict flow of blood therethrough to the aneurysm while the stent device is positioned within the blood vessel, and
wherein the open cell geometry is configured to be positioned so as to maintain blood flow through to the at least one side-branch while the stent device is positioned within the blood vessel.

13. A device as in claim 12, wherein the occlusional cell geometry has smaller cells than the open cell geometry.

14. A device as in claim 12, wherein the occlusional cell geometry comprises approximately 10-90 percent of the frame.

15. A device as in claim 14, wherein the occlusional cell geometry comprises approximately 30-40 percent of the frame.

16. A device as in claim 12, wherein the occlusional cell geometry is disposed approximately equidistant from the ends.

17. A device as in claim 12, wherein the frame includes at least one anchoring portion which provide radial anchoring force.

18. A method of covering an aneurysm in a blood vessel wherein the blood vessel includes at least one side-branch near the aneurysm, the method comprising:
advancing a stent through the blood vessel, wherein the stent comprises a tubular frame having a first end, a second end, an open cell geometry and a covering; and
positioning the stent within the blood vessel so that the covering substantially covers the aneurysm restricting blood flow to the aneurysm and the open cell geometry substantially covers the at least one side-branch allowing blood flow to the at least one side-branch.

19. A method as in claim 18, wherein the covering is disposed approximately equidistant from the ends and positioning comprises positioning the ends on opposite sides of the aneurysm.

20. A method as in claim 18, wherein the stent includes an anchoring portion near the first end and the covering near the second end and wherein the blood vessel includes a bifurcation near the aneurysm, and wherein positioning comprises positioning the anchoring portion within the blood vessel so as to anchor the stent while the second end is disposed near the bifurcation.

21. A method as in claim 18, wherein positioning the stent comprises expanding the stent within the blood vessel.

22. A method as in claim 18, wherein the blood vessel comprises a cerebral blood vessel.

23. A method as in claim 18, further comprising altering the open cell geometry.

24. A method as in claim 23, wherein altering comprises expanding an inflatable member within a cell of the open cell geometry so as to widen the cell.
